(19) United States
(12) Patent Application Publication
(10) Pub. No.: US 2026/0027037 A1
(43) Pub. Date: Jan. 29, 2026

(54) COSMETIC COMPOSITION CONTAINING LOW MOLECULAR WEIGHT ANIONIC PEPTIDE ISOLATED FROM CITRUS JUNOS SEED

(71) Applicants: COSEEDBIOPHARM CO.,LTD., Cheongju-si, Chungcheongbuk-do (KR); COSMECCA KOREA CO., LTD., Chungcheongbuk-do (KR)

(72) Inventors: Su An SIM, Cheongju-si (KR); Jung No LEE, Cheongju-si (KR); Sung Min PARK, Cheongju-si (KR); Hyun Ju KOH, Jeju-si (KR); Hyun Dae CHO, Cheongju-si (KR); Jae Yong SEO, Cheongju-si (KR); A Reum JANG, Seoul (KR); Seung Ji JEONG, Seongnaim-si, Gyeonggi-do (KR)

(73) Assignees: COSEEDBIOPHARM CO.,LTD., Cheongju-si, Chungcheongbuk-do (KR); COSMECCA KOREA CO., LTD., Chungcheongbuk-do (KR)

(21) Appl. No.: 18/996,198

(22) PCT Filed: Dec. 21, 2023
(86) PCT No.: PCT/KR2023/021277

§ 371 Date: Jan. 17, 2025

(30) Foreign Application Priority Data
Dec. 23, 2022 (KR) 10-2022-0183097

(51) Int. Cl. A61K 8/9789; A61K 8/34; A61Q 17/00; A61Q 19/00; A61Q 19/08

(52) U.S. Cl. CPC A61K 8/9789; A61K 8/345; A61Q 17/00; A61Q 19/005; A61Q 19/08; A61K 2800/805; A61K 2800/84

(57) ABSTRACT

The present invention relates to a cosmetic composition containing a low-molecular weight anionic peptide isolated from Citrus junos seeds. More specifically, the present invention relates to a method for isolating a low-molecular weight peptide from Citrus junos seeds and processing same into a low-molecular weight anionic peptide, and a cosmetic composition including same as an active ingredient.

### TECHNICAL FIELD

The present invention relates to a cosmetic composition containing a low-molecular-weight anionic peptide isolated from Yuja (Citrus junos) seeds. More specifically, the present invention relates to a method of preparing a cosmetic composition including isolating a low-molecular weight peptide from Citrus junos seeds and converting the peptide into a low-molecular weight anionic peptide, and a cosmetic composition containing the same as an active ingredient.

### BACKGROUND ART

It is known that countless pollutants in the air generated by combustion in automobiles, boilers, power plants, and the like and fine dust emitted therefrom cause various diseases and damage to the skin barrier, causing inflammation, trouble, and accelerated skin aging. The term “fine dust” generally refers to any pollutant with a very small size. Fine dust particles cannot be detected with the naked eye because they are less than 10 μm in diameter, and the fine dust particles are not blocked by the skin and penetrate because they are only one-twentieth of the size of skin pores. Fine dust particles cannot be easily removed and cause various chemical stimuli to keratinocytes and lipid membranes, thus leading to inflammation and thus decreased skin immunity, acne, dry skin, pigmentation, destruction of the skin barrier, skin wrinkles and the like.

In general, the external surface of substances such as yellow dust, pollution, and fine dust are affected by heavy metals such as cadmium, lead, and arsenic, and thus are negatively charged. Therefore, research is being conducted on substances that can prevent the adhesion of fine dust or can remove fine dust based on electrostatic repulsion. However, screened materials based on simple natural plant extracts are used or developed into formulation ingredients, and thus the effects thereof are insufficient.

Meanwhile, the skin is anionic due to the phospholipid components present in the stratum corneum thereof. However, no active research has been conducted on technologies that easily remove pollutants from the skin and address skin troubles using these anions and the electrical power of anions.

Accordingly, in the present invention, rather than simply finding a negatively charged material among natural plant extracts, high-quality low-molecular-weight peptides are converted into anionic peptides by hydrolysis of various materials. As a result, proteins are extracted from Citrus junos seeds, which are typically discarded as a by-product, are hydrolyzed into low-molecular-weight peptides to increase skin absorption rate, and are isolated into negatively charged peptides to develop a material for preventing the attachment of fine dust such as yellow dust and air pollution, and alleviating skin irritation and wrinkles.

### DISCLOSURE

### Technical Problem

Therefore, it is an object of the present invention to develop and provide a cosmetic composition that prevents the attachment of fine dust such as yellow dust and air pollution and alleviates skin damage and wrinkles caused thereby by separating a functional low-molecular-weight peptide from a naturally-derived material and then separating a negatively charged peptide from the peptide.

### Technical Solution

In accordance with one aspect of the present invention, provided is a cosmetic composition containing a low molecular weight anionic peptide obtained by a process including (a) immersing a Citrus junos seed powder in purified water, (b) inducing a primary enzymatic reaction using a subtilisin enzyme derived from Bacillus subtilis after the immersion in step (a), (c) inducing a secondary enzymatic reaction using pepsin after the primary enzymatic reaction in step (b), (d) centrifuging the enzymatic reaction solution to recover a supernatant after the secondary enzymatic reaction in step (c), (e) adding ethanol to the supernatant recovered in step (d) to induce a precipitation reaction and centrifuging the precipitate to obtain a supernatant, and (f) separating a low-molecular-weight anionic peptide from the supernatant obtained in step (e) or a solution obtained by dissolving a powder obtained by lyophilizing the supernatant in distilled water using a cation exchange resin.

In the cosmetic composition of the present invention, the low-molecular-weight anionic peptide derived from the Citrus junos seed may have a molecular weight of 1,000 Da or less.

In the cosmetic composition of the present invention, the primary enzymatic reaction of step (b) may be performed at a pH of 6 to 8, and the secondary enzymatic reaction of step (c) may be performed at a pH of 2 to 4.

In the cosmetic composition of the present invention, the primary enzymatic reaction of step (b) may be performed at a pressure of 20 to 100 bar and a temperature of 50 to 60° C., and the secondary enzymatic reaction of step (c) may be performed at a pressure of 20 to 100 bar and a temperature of 35 to 45° C.

In the cosmetic composition of the present invention, the cosmetic composition may be used to prevent fine dust adsorption.

In the cosmetic composition of the present invention, the cosmetic composition may be used to alleviate skin irritation.

In the cosmetic composition of the present invention, the cosmetic composition may be used to alleviate skin wrinkles.

### Advantageous Effects

The present invention provides a method of preparing low-molecular-weight peptides from Citrus junos seeds and separating the peptides depending on charge, and a cosmetic composition containing a low-molecular-weight anionic peptide.

In addition, the cosmetic composition according to the present invention prevents fine dust such as yellow dust from adhering to the skin and pollution, ameliorates skin irritation caused by an increase in inflammatory cytokines, and relieves skin wrinkles.

### BRIEF DESCRIPTION OF THE DRAWING

FIG. 1 shows the surface potential of low-molecular-weight peptides derived from Citrus junos seeds and the low-molecular-weight anionic peptide derived from Citrus junos seeds.

FIG. 2 shows the molecular weights of low-molecular-weight peptides derived from Citrus junos seeds and low-molecular-weight anionic peptides derived from Citrus junos seeds.

FIG. 3 shows the efficacy of suppressing nitrogen oxide due to fine dust such as yellow dust and air pollution of the low-molecular-weight peptide derived from Citrus junos seeds and the low-molecular-weight anionic peptide derived from Citrus junos seeds.

FIG. 4 shows the MMP-1 inhibition efficacy of low-molecular-weight peptides derived from Citrus junos seeds and low-molecular-weight anionic peptides derived from Citrus junos seeds.

FIG. 5 shows the effects of the cosmetic composition containing low-molecular-weight anionic peptides derived from Citrus junos seeds on prevention of absorption of fine dust such as yellow dust and air pollution.

FIG. 6 shows the effects of the cosmetic composition containing low-molecular-weight anionic peptides derived from Citrus junos seeds on suppression of expression of inflammatory cytokines caused by fine dust such as yellow dust and air pollution.

### BEST MODE

Citrus junos is an evergreen broadleaf shrub of the Rutaceae family, is a type of citrus plant, is mainly found in East Asia, was identified in Korea in the Silla Dynasty and has been cultivated in the southern region. Meanwhile, Citrus junos seeds account for 10 to 20 wt % of the weight of the Citrus junos fruit flesh, are rich in active ingredients such as limonoids and bioflavonoids, and contain great amounts of antioxidant substances, thus being developed into various functional materials. However, various active ingredients of Citrus junos seeds are mixed with great amounts of lipids, and thus are limitedly used in oils containing fat components, and taste bitter, thus being mostly discarded as byproducts after preparation. In addition, although Citrus junos seeds contain about 20 to 30 wt % of fats and oils and about 10 to 20 wt % of crude protein, there are few use cases for Citrus junos seeds. Accordingly, the present inventors determined that a novel functional material with excellent physiological activity, safety, and stability can be developed by extracting proteins contained in large quantities in Citrus junos seeds wasted as by-products, and decomposing the proteins into peptides, which are amino acid polymers with excellent skin absorption rate due to smaller sizes thereof than proteins, and utilizing the same.

In one aspect, the present invention provides a cosmetic composition containing a low molecular weight anionic peptide obtained by a process including (a) immersing a Citrus junos seed powder in purified water, (b) inducing a primary enzymatic reaction using a subtilisin enzyme derived from Bacillus subtilis after the immersion in step (a), (c) inducing a secondary enzymatic reaction using pepsin after the primary enzymatic reaction in step (b), (d) centrifuging the enzymatic reaction solution to recover a supernatant after the secondary enzymatic reaction in step (c), (e) adding ethanol to the supernatant recovered in step (d) to induce a precipitation reaction centrifuging the precipitate to obtain a supernatant, and (f) separating a low-molecular-weight anionic peptide from the supernatant obtained in step (e) or a solution obtained by dissolving a powder obtained by lyophilizing the supernatant in distilled water using a cation exchange resin.

Below, each step of the process of preparing the cosmetic composition containing low-molecular-weight anionic peptides derived from Citrus junos seeds of the present invention will be described in detail.

### Step (a): Immersing Citrus junos Seed Powder

This step is a process of immersing a yuja (Citrus junos) seed powder in purified water.

Citrus junos seeds are ground using a grinder to obtain a Citrus junos seed powder and then the obtained Citrus junos seed powder is immersed in purified water.

### Step (b): Primary Enzymatic Reaction

This step is a process of inducing a primary enzymatic reaction using a subtilisin enzyme derived from Bacillus subtilis after the immersion in step (a).

In this step, the Citrus junos seed powder immersed in purified water in step (a) is subjected to the primary enzymatic reaction. At this time, the primary enzymatic reaction is preferably performed at a pH of 6 to 8, and the pH of the primary enzymatic reaction solution is adjusted to 6 to 8 to optimize the enzymatic reaction.

The primary enzymatic reaction in this step is performed using an enzyme derived from Bacillus subtilis, preferably subtilisin. Specifically, the primary enzymatic reaction of this step may be performed using an ultra-high pressure enzyme reactor provided with a polymer coated with subtilisin, a protein-decomposing enzyme isolated from Bacillus subtilis, and is preferably performed at a pressure of 20 to 100 bar and a temperature of 50 to 60° C. At this time, the reaction time is preferably about 12 hours to about 36 hours.

### Step (c): Secondary Enzymatic Reaction

This step is a process of inducing a secondary enzymatic reaction using pepsin after the primary enzymatic reaction of step (b).

In this step, after the primary enzymatic reaction, the solution is recovered from the reactor and treated with pepsin to induce a secondary enzymatic reaction. At this time, the secondary enzymatic reaction is preferably performed at a pH of 2 to 4. The pH of the secondary enzymatic reaction solution is adjusted to 2 to 4 to optimize the enzymatic reaction.

The secondary enzymatic reaction of this step is preferably performed in an ultra-high pressure enzyme reactor provided with a polymer coated with pepsin and is preferably performed at a pressure of 20 to 100 bar and a temperature of 35 to 45° C. At this time, the reaction time is preferably 3 to 9 hours.

After the secondary enzymatic reaction in this step, the solution obtained from the secondary reaction is preferably boiled to inactivate the excess enzyme.

### Step (d): Recovery of Supernatant

This step is a process of recovering the supernatant by centrifugation after the secondary enzymatic reaction of step (c). In other words, the solution in which the secondary enzymatic reaction of step (c) is completed is centrifuged to recover the supernatant.

### Step (e): Obtaining Supernatant After Ethanol Precipitation

This step is a process of adding ethanol to the supernatant recovered in step (d) to induce a precipitation reaction and then centrifuging the precipitate to obtain the supernatant.

In this step, ethanol is added to the supernatant recovered in step (d) to induce ethanol precipitation. A ratio of the ethanol to the supernatant may be 1:1. When ethanol is added in this way, precipitation is induced and the precipitate settles to the bottom. The ethanol precipitation is preferably performed at a refrigerated temperature.

After the ethanol precipitation reaction is induced as described above, centrifugation is performed to obtain the supernatant. In this case, the supernatant obtained by the centrifugation is preferably filtered, preferably through a 0.1 to 0.5 μm precision filter membrane, to obtain a filtrate. After filtering, the filtrate is lyophilized if necessary.

### Step (f): Obtaining Low-Molecular-Weight Anionic Peptide

This step is a process of separating a low-molecular-weight anionic peptide from the supernatant obtained in step (e) or a solution obtained by dissolving a lyophilized powder of the supernatant in distilled water using a cation exchange resin after step (e).

More specifically, the low-molecular-weight anionic peptide of step (f) of the present invention may be obtained by separation from the supernatant as follows.

The cation exchange resin is swollen in a 5 to 10-fold 1N HCl aqueous solution and then packed into an open column tube. Then, the peptide is activated by flowing a 5- to 10-fold 1M HCl aqueous solution and then is washed with 5- to 10-fold distilled water. Then, the supernatant obtained in step (e) or the solution obtained by dissolving a lyophilized powder of the supernatant in distilled water is loaded (up to 20 g/L resin volume) into an open column and then 5 to 10-fold distilled water is continuously flowed at an appropriate rate to induce elution. The eluent thus obtained may be lyophilized to produce a powder.

After step (f), a low-molecular-weight anionic peptide having a molecular weight of 1,000 Da or less (a low-molecular-weight anionic peptide having a negative surface potential) may be obtained. Preferably, size exclusion chromatography, or the like may be further performed to obtain a low-molecular-weight anionic peptide having a molecular weight of 500 Da or less.

Meanwhile, the cation exchange resin used in step (f) is preferably prepared according to the following steps (a) to (e).

### (a) Agarose Bead Swelling

This step is a process of swelling agarose beads in an aqueous NaCl solution and then filtering the result.

Preferably, agarose beads, which are biopolymers, are swollen in a 5-to 10-fold 1N aqueous NaCl solution and then filtered.

### (b) Agarose Bead Activation

This step is a process of activating the agarose filtered in step (a) in an aqueous NaOH solution while stirring. At this time, preferably, the filtered agarose is activated in a 5-to 10-fold larger 5N aqueous NaOH solution for about 30 minutes while stirring.

### (c) Preparation of Cation Exchange Resin

This step is a process of preparing a cation exchange resin by adding glycidyltrimethylammonium chloride to the agarose activated in step (b), following by allowing reaction. At this time, the cation exchange resin is preferably prepared by stirring at 50 to 60° C. for 2 to 4 hours.

### (d) Cation Exchange Resin Washing

This step is a process of washing the cation exchange resin by adding distilled water after step (d). The cation exchange resin is preferably washed by adding 5-to 10-fold distilled water, allowing the mixture to stand and then removing the supernatant 3 to 5 times.

### (e) pH Adjustment

This step is a process of adjusting the pH to 6.5 to 7.5 after step (d). Meanwhile, the washing may be further performed 1 to 3 times as needed before filtration.

As such, the cation exchange resin for separating low-molecular-weight anionic peptide of the present invention may be finally prepared by performing steps (a) to (e).

Meanwhile, the present invention provides a cosmetic composition containing the low-molecular-weight anionic peptide obtained through a process including steps (a) to (f).

In the cosmetic composition of the present invention, the cosmetic composition may be used to prevent fine dust adsorption.

In the cosmetic composition of the present invention, the cosmetic composition may be used to alleviate skin irritation.

In the cosmetic composition of the present invention, the cosmetic composition may be used to alleviate skin wrinkles.

Meanwhile, in the cosmetic composition of the present invention, the effect of preventing fine dust adsorption or alleviating skin irritation or alleviating skin wrinkles may be obtained by inhibition of nitric oxide production caused by fine dust such as yellow dust or air pollution or inhibition of inflammatory cytokine expression.

In the cosmetic composition of the present invention, the low molecular weight anionic peptide of the present invention is preferably contained in an amount of 0.01 to 30.0 wt % based on the total weight of the cosmetic composition.

In the cosmetic composition of the present invention, the cosmetic composition may be, for example, any one formulation selected from the group consisting of a toner, a gel, a water-soluble liquid, a cream, an essence, an oil-in-water (O/W) type cosmetic, a water-in-oil (W/O) type cosmetic, an ointment, a foundation, a skin cover, a lipstick, and a scalp cosmetic.

The cosmetic composition of the present invention may be prepared into any formulation commonly prepared in the art, for example, a toner, lotion, cream, essence, tonic water, cleanser, shampoo, treatment, curl cream, hair gel, or the like, but is not limited thereto.

Meanwhile, all technical terms used herein, unless otherwise defined, have the following definitions and correspond to the meanings generally understood by those skilled in the art in the relevant field of the present invention. In addition, although preferred methods or reagents are described in this specification, similar or equivalent ones are also within the scope of the present invention.

It will be further understood that terms such as “comprises” and “comprising”, when used herein, specify the presence of suggested steps or components or a group of steps or components, but do not preclude the presence or addition of other steps or components or group of steps or components unless otherwise mentioned.

Hereinafter, the present invention will be described in more detail with reference to the following Examples and Experimental Examples. However, the scope of the present invention is not limited to the following Examples and Experimental Examples, but includes all modifications of technical ideas equivalent thereto.

### Example 1: Production of Low-Molecular-Weight Peptide Derived From Citrus junos Seeds

Citrus junos seeds were ground using a grinder to obtain a Citrus junos seed powder. The obtained Citrus junos seed powder was immersed in purified water to adjust the pH to 7 and then an enzymatic decomposition reaction was performed in an ultra-high pressure enzyme reactor provided with a polymer coated with a subtilisin enzyme derived from Bacillus subtilis at a pressure of 100 bar and a temperature of 55° C. for 24 hours. Then, the solution was recovered from the reactor, the pH of the solution was adjusted to 3, and an enzymatic decomposition reaction was performed in an ultra-high pressure enzyme reactor provided with a polymer coated with pepsin at a pressure of 100 bar and a temperature of 40° C. for 6 hours, and then heated for 30 minutes to inactivate the excess enzyme and terminate the reaction. Then, the solution was recovered from the reactor and centrifuged to obtain a supernatant. Ethanol was added at a weight ratio of 1:1 based on the weight of the supernatant and the supernatant excluding the precipitate was centrifuged. Then, the result was allowed to pass through a 0.2 μm precision filtration membrane and the obtained filtrate was lyophilized to prepare a low-molecular-weight peptide powder of Citrus junos seeds.

### Example 2: Production of Low-Molecular-Weight Anionic Peptides Derived From Citrus junos Seeds

### (1) Production of Cation Exchange Resin (Affinity Resin) for Separating Anionic Peptide

First, an affinity resin for separating anionic peptide was prepared in order to prepare low-molecular-weight anionic peptides from Citrus junos seeds.

Agarose beads, a biopolymer, were swollen in a 10-fold 1N aqueous NaCl solution and then filtered, and the filtered agarose was activated in a 10-fold 5N aqueous NaOH solution for 30 minutes while stirring. Glycidyltrimethylammonium chloride was added to the activated agarose, followed by reaction at 55° C. for 3 hours. Then, a washing process including adding 10-fold distilled water, allowing the result to stand, and removing the supernatant was performed four times, the pH was adjusted to 7, the washing was performed twice more and then filtration was performed.

### (2) Production of Low-Molecular-Weight Anionic Peptide From Citrus junos Seeds

The cation exchange resin for separating anionic peptide produced in Example 2-(1) was swollen in a 1N HCl aqueous solution 10 times the volume of the resin and packed in an open column tube. A 1M HCl aqueous solution in an amount of 10 times the volume of the resin was allowed to flow through the resin to activate the resin and then distilled water in an amount of 10 times the volume of the resin was allowed to flow to wash the resin.

Meanwhile, the dried Citrus junos seed low-molecular-weight peptide powder produced in Example 1 was dissolved in distilled water and was loaded in a predetermined amount (maximum 20 g/L resin volume) into an open column, and then was eluted by flowing distilled water 10 times the volume of the resin at an appropriate rate. The eluted solution was lyophilized to produce Citrus junos seed low-molecular-weight anionic peptide powder.

### Comparative Example 1: Production of Citrus junos Seed Low-Molecular-Weight Anionic Peptide Using Strong Acid Cation Exchange Resin

In Comparative Example 1, Citrus junos seed low-molecular-weight anionic peptide was produced using a commercially available strong acid cation exchange resin, a TRILITE MC-08 resin.

The TRILITE MC-08 resin was each swollen in 5 times the volume of 1 M HCl aqueous solution and packed in an open column tube. Then, the resin was activated by flowing a 1M aqueous HCl solution in an amount of 10 times the volume of the resin and then was washed with a distilled water in an amount of 10 times the volume of the resin.

Then, in the same manner as in Example 2, the dried Citrus junos seed low-molecular-weight peptide powder produced in Example 1 was dissolved in distilled water and was loaded in a predetermined amount (maximum 20 g/L resin volume) into an open column, and then was eluted by flowing distilled water 10 times the volume of the resin at an appropriate rate. The eluted solution was lyophilized to produce Citrus junos seed low-molecular-weight anionic peptide powder of Comparative Example 1.

### Comparative Example 2: Production of Citrus junos Seed Low-Molecular-Weight Anionic Peptide Using Weak Acid Cation Exchange Resin

In Comparative Example 2, Citrus junos seed low-molecular-weight anionic peptide was produced using a commercially available weak acid cation exchange resin, DIAON WK60L.

The DIAON WK60L resin was each swollen in 5 times the volume of 1 M HCl aqueous solution and packed in an open column tube. Then, the resin was activated by flowing a 1M HCl aqueous solution in an amount of 10 times the volume of the resin and then was washed with a distilled water in an amount of 10 times the volume of the resin.

Then, in the same manner as in Example 2, the dried Citrus junos seed low-molecular-weight peptide powder produced in Example 1 was dissolved in distilled water, was loaded in a predetermined amount (maximum 20 g/L resin volume) into an open column, and then was eluted by flowing distilled water 10 times the volume of the resin at an appropriate rate. The eluted solution was lyophilized to produce Citrus junos seed low-molecular-weight anionic peptide powder of Comparative Example 2.

### Experimental Example 1: Surface Potential Measurement

In this experimental example, the surface potential measurement experiments of Examples 1 and 2, and Comparative Examples 1 and 2 were conducted. In order to analyze the surface charge, the surface charge of each peptide was determined using a zeta potential analyzer, which is a measuring device based on dynamic light scattering and electrophoretic light scattering, and the measurement was performed in triplicate and the three measurement values were averaged.

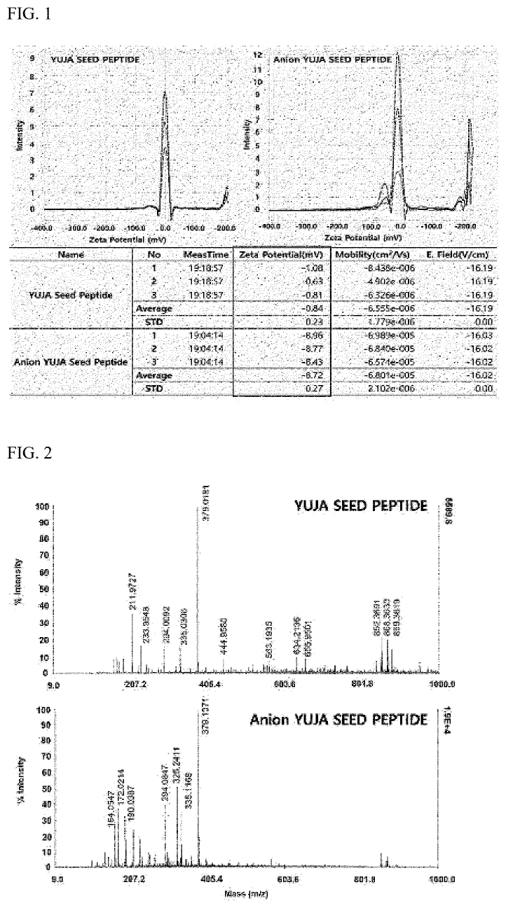

FIG. 1 shows the surface potential of the citron seed low-molecular-weight peptide (Example 1) and the citron seed low-molecular-weight anionic peptide (Example 2). As can be seen from FIG. 1, the zeta potential of Example 1 was measured as −0.84 mV and the zeta potential of Example 2 was measured as −8.72 mV, which indicates that the separation of the low-molecular-weight anionic peptide was well performed in Example 2 using the cation exchange resin (affinity resin) of the present invention.

In addition, the zeta potential of Comparative Example 1 was measured as −3.25 mV and the zeta potential of Comparative Example 2 was measured as −4.82 mV, which indicates that the separation of low-molecular-weight anionic peptides using the cation exchange resin (affinity resin) for separating anionic peptide produced in Example 2 caused a higher separation ability than the separation of low-molecular-weight anionic peptides using the commercially available strongly acidic and weakly acidic cation exchange resins of Comparative Examples 1 and 2.

### Experimental Example 2: Molecular Weight Measurement

In this experimental example, the molecular weight measurement experiments of Examples 1 and 2 were conducted. For molecular weight measurement, a MALDI-TOF mass spectrometer (MALDI-TOF MS) was used.

Examples 1 and 2 were dissolved in a 50% (v/v) methanol/water mixture and then 1 μl of the mixture was mixed with 1 μl of a sinapinic acid (SA) matrix (50% (v/v) 0.1% TFA acetonitrile/50% (v/v) water). Then, the mixture was spotted on a stainless steel MALDI plate and dried at room temperature. The analysis was performed using a Microflex LRF MALDI-TOF mass spectrometer and Bruker UltrafleXtreme (Bruker Daltonics, Bremen, Germany), and the intensity of each ion was calculated from a sum of the first isotope peak area and the third isotope peak area. Spectrum acquisition and processing were performed using FlexAnalysis software (ver. 3.3, Bruker Daltonics, Bremen, Germany), and the results are shown in FIG. 2.

FIG. 2 shows the molecular weights of the low-molecular-weight peptide and low-molecular-weight anionic peptide of Citrus junos seeds. The molecular weight distributions of Examples 1 and 2 were found to be about 1,000 Da or less, more specifically, about 500 Da or less. This means that Examples 1 and 2 were well hydrolyzed into low-molecular-weight peptides.

### Experimental Example 3: Confirmation of Inhibition Rate of NO Caused by Fine Dust

In this experimental example, an experiment was conducted to determine the effect of Examples 1 and 2 on inhibition of nitric oxide (NO) generated by fine dust stimulation.

RAW 264.7 cells, which are mouse-derived macrophages, were seeded at 1×10⁵ cells/well in a 96-well plate, stabilized in a 37° C. and 5% CO₂ cell incubator for one day, and then treated with 200 μg/ml of fine dust. Each sample was diluted to a concentration of 200 μg/ml and then treated with 5 μM nordihydroguaiaretic acid (NDGA) as a positive control and then reacted for 24 hours, 100 μl of the separated culture supernatant was added to the same amount of Griess reagent and then reacted at room temperature, and then the absorbance was measured at 540 nm using a spectrophotometer (microplate reader). The concentration of nitric oxide was calculated by comparison with the standard curve obtained using sodium nitrite (NaNO₂).

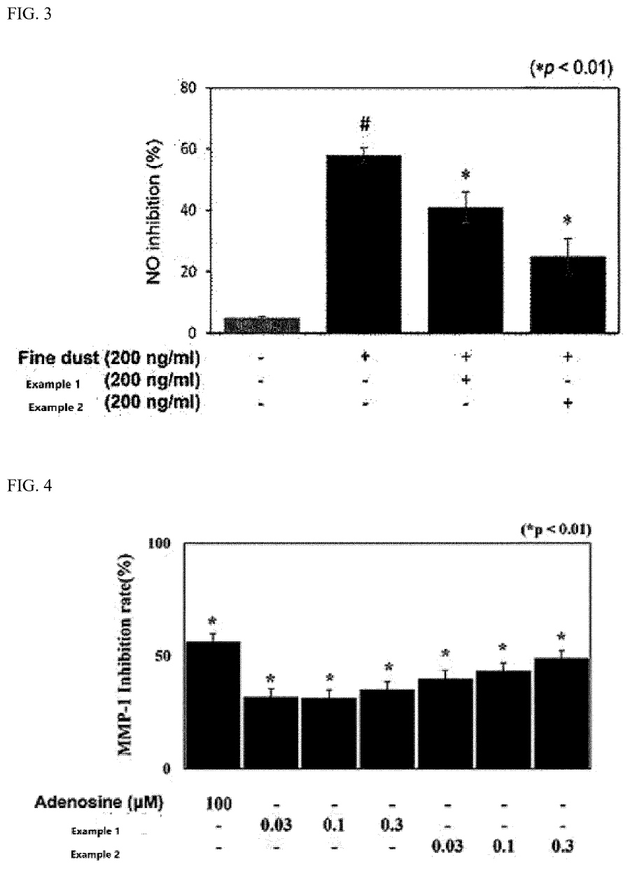

FIG. 3 shows the inhibitory effect of nitrogen oxides caused by fine dust such as yellow dust and air pollution by low-molecular-weight peptides and low-molecular-weight anionic peptides derived from Citrus junos seeds. As can be seen from FIG. 3, the NO production inhibition activity was higher when treated with Example 2 compared to when treated with NDGA or Example 1. In other words, Example 1 (Citrus junos seed low-molecular-weight peptide) also exhibited an NO production inhibition effect, whereas Example 2 (Citrus junos seed low-molecular-weight anionic peptide) separated depending on charge exhibited better NO production inhibition activity.

### Experimental Example 4: Measurement of MMP-1 Production Inhibition Effect

In this experimental example, an experiment was conducted to determine MMP-1 (matrix metalloproteinase-1) production inhibition effects of Examples 1 and 2.

Human dermal fibroblasts (HDFn) were seeded at 5×10⁴ cells/well in 24-well plates, were stabilized in a 37° C., 5% CO₂ cell incubator for one day and then treated with 1 J/cm² of UVA. Each sample was diluted by concentration, treated with a medium containing 2% FBS, treated with adenosine (raw material for wrinkle functional notification) at a concentration of 400 μM as a positive control group and reacted for 48 hours. The MMP-1 expression level of the supernatant was determined using an R&D systems kit (DY901) and the pellet was treated with a MTT solution to determine cytotoxicity.

After the MMP-1 ELISA, the absorbance was measured at a wavelength of 450 nm to calculate the amount of MMP-1 produced, the MMP-1 expression inhibition rate (%) compared to the control group was calculated, and the calculation is based on the following equation:

$$MMP - 1 production inhibition rate ( \% ) = 1 - \frac{Amount of MMP - 1 of Experimental group}{Amount of MMP - 1 of Control group} \times 100 \qquad [ Equation 1 ]$$

FIG. 4 shows the MMP-1 inhibitory effect of low-molecular-weight peptides and low-molecular-weight anionic peptides derived from Citrus junos seeds. As can be seen from FIG. 4, the MMP-1 production inhibition activity was higher when treated with Example 2 compared to when treated with Example 1. In other words, Example 1 (Citrus junos seed low-molecular-weight peptide) also exhibited MMP-1 production inhibition activity, whereas Example 2 (Citrus junos seed low-molecular-weight anionic peptide) separated depending on charge exhibited better MMP-1 production inhibition activity.

### Preparation Example 1: Preparation of Formulations Using Examples 1 and 2

In this Preparation Example, cosmetic compositions were prepared using Preparation Examples 1 and 2 using the peptide of Example 1 (Citrus junos seed low-molecular peptide) or Example 2 (Citrus junos seed low-molecular anionic peptide) and Comparative Example 3 using neither Example 1 nor Example 2, in accordance with the compositions shown in Table 1 below.

TABLE 1

| | Comparative | Preparation | Preparation |
|---|---|---|---|
| | Example 3 | Example 1 | Example 2 |
| Ingredient | (wt %) | (wt %) | (wt %) |
| Example 1 | 0 | 5 | 0 |
| Example 2 | 0 | 0 | 5 |
| EDTA-2Na | 0.02 | 0.02 | 0.02 |
| Cetostearyl alcohol | 2.0 | 2.0 | 2.0 |
| Glyceryl stearate | 1.5 | 1.5 | 1.5 |
| Microcrystalline | 0.7 | 0.7 | 0.7 |
| Squalane | 5.0 | 5.0 | 5.0 |
| Liquid paraffin | 3.0 | 3.0 | 3.0 |
| Trioctanoin | 5.0 | 5.0 | 5.0 |
| Polysorbate | 1.2 | 1.2 | 1.2 |
| Sorbitan stearate | 0.5 | 0.5 | 0.5 |
| Tocopheryl acetate | 0.2 | 0.2 | 0.2 |
| Cyclomethicone | 3.0 | 3.0 | 3.0 |
| BHT | 0.05 | 0.05 | 0.05 |
| Fragrance, | Appropriate | Appropriate | Appropriate |
| Preservatives | amount | amount | amount |
| Purified water | to 100 | to 100 | to 100 |

### Experimental Example 5: Measurement of Effect of Preventing Fine Dust Adsorption

In this experimental example, the effects of preventing fine dust adsorption of the cosmetic compositions of Preparation Example 1 (using Example 1), Preparation Example 2 (using Example 2), and Comparative Example 3 (neither using Example 1 nor Example 2) were determined. To this end, carbon black having a size and surface potential similar to fine dust was used.

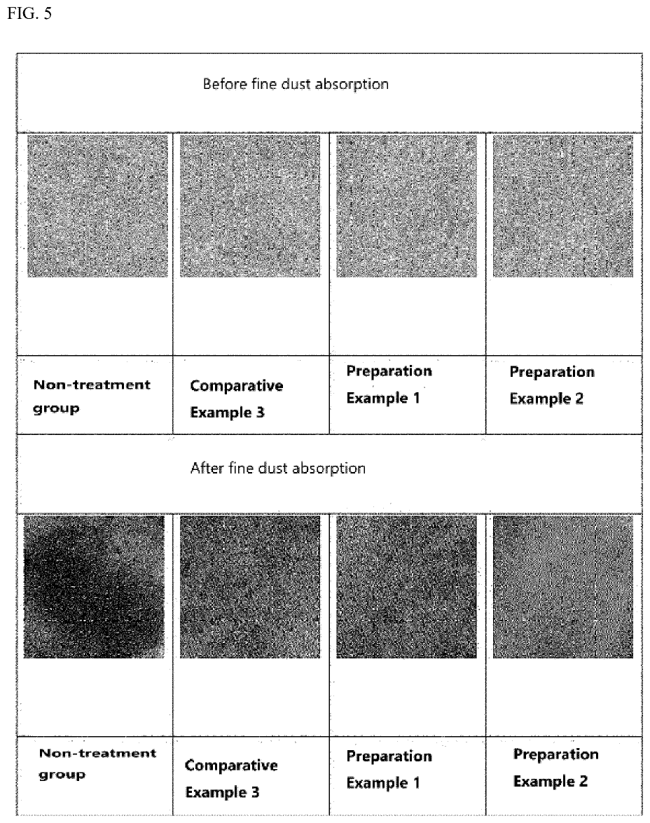

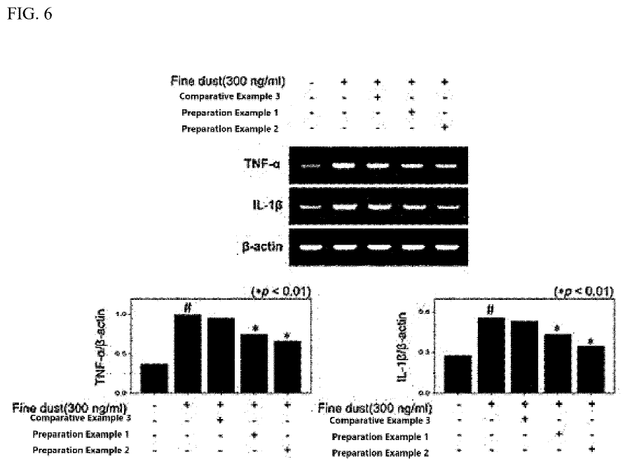

In order to create the state of fine dust floating in the air, an appropriate amount of carbon black was uniformly sprayed within the chamber using a fine dust floating chamber equipped with a propeller. The adsorption effect experiment was conducted by applying Comparative Example 3, and Preparation Example 1 and Preparation Example 2 onto areas of the forearm area, naturally drying the areas, imaging test areas using a high-resolution digital camera and Folliscope (enlarged image of the skin surface), and analyzing the brightness using an image analysis program to obtain the change before and after fine dust adsorption (FIG. 5). Meanwhile, the amount of adsorbed fine dust decreases as the brightness increases. The effect of preventing fine dust adsorption was determined to be present when the adsorption amount (Δ) of group treated with the test product decreased statistically significantly (p<0.05), compared to the adsorption amount (Δ) of the group untreated therewith.

The amount of adsorbed fine dust was analyzed to evaluate the improvement rate (%) of Comparative Example 3 and Preparation Examples 1 and 2 compared to the untreated group, and the results are shown in Table 2 and FIG. 5.

$$Improvement rate ( \% ) = \frac{\begin{matrix}Amount of adsorbed fine dust \\ of treated group ( Δ ) - \\ amount of adsorbed fine dust \\ of untreated group ( Δ )\end{matrix}}{\begin{matrix}Amount of adsorbed fine \\ dust of untreated group ( Δ )\end{matrix}} \times 100 \qquad [ Equation 2 ]$$

TABLE 2

| | Comparative | Preparation | Preparation |
|---|---|---|---|
| | Example 3 | Example 1 | Example 2 |
| Ratio of brightness before fine | 78.8 | 78.5 | 68.1 |
| dust absorption to brightness after | | | |
| fine dust absorption (%) | | | |
| Improvement (%) | 2.1 | 3.5 | 8.3 |

As can be seen from Table 2 and FIG. 5, the brightness of Preparation Example 2 using the low-molecular-weight anionic peptide from Citrus junos seeds (Example 2) was 68.1% and the improvement rate was 8.3%, which indicates that the amount of adsorbed fine dust was significantly improved compared to the cosmetic compositions of Preparation Example 1 (using Example 1) and Comparative Example 3 (control group).

### Experimental Example 6: Confirmation of Expression of Inflammatory Cytokines Caused by Fine Dust

In this experimental example, the effects of the cosmetic compositions of Preparation Example 1 (using Example 1), Preparation Example 2 (using Example 2), and Comparative Example 3 (using neither Example 1 nor Example 2) on inhibition of the expression of inflammatory cytokines generated by fine dust stimulation were determined.

For the experiment, Sebutape was attached to the lower extremities of 30 subjects (women aged 20 to 35) before applying fine dust, and cytokines on the skin surface were obtained. This process was performed a total of 5 times, each cytokine was immersed in a glass vial containing 1 ml of PBS buffer and then sonicated for 1 hour, and then the cytokine content was measured.

Then, the fine dust was mixed with mineral oil at a ratio of 1:1, the resulting mixture was applied to the lower extremities, and Comparative Example 3, and Preparation Example 1 and Preparation Example 2 were applied to perform the Sebutape patch. After 24 hours, the patch was removed and the cytokines were obtained in the same manner as the cytokine obtaining process before application. The cytokine content was measured using an ELISA kit, the total protein content was measured using a protein quantification kit, and the cytokine content was corrected based on the total protein content to analyze the results (FIG. 6).

As can be seen from FIG. 6, the cosmetic compositions of Preparation Example 1 using the low-molecular-weight peptide derived from Citrus junos seeds (Example 1) and Preparation Example 2 using the low-molecular-weight anionic peptide derived from Citrus junos seeds (Example 2) significantly reduced the expression levels of inflammatory cytokines (TNF-α, IL-1β) due to fine dust compared to Comparative Example 3, and it can be seen that Preparation Example 2 using the low-molecular-weight anionic peptide derived from Citrus junos seeds (Example 2) exhibited a particularly excellent effect. That is, it can be seen that a cosmetic composition with an excellent skin irritation relief effect can be prepared using the low-molecular-weight anionic peptide derived from Citrus junos seeds of the present invention.

### Experimental Example 7: Measurement of Effect of Alleviating Wrinkles

In this experimental example, the effects of the cosmetic compositions of Preparation Example 1 (using Example 1), Preparation Example 2 (using Example 2), and Comparative Example 3 (neither using Example 1 nor Example 2) on alleviation of skin wrinkles were determined.

Therefore, the effects of the cosmetic compositions of Preparation Example 1 (using Example 1), Preparation Example 2 (using Example 2), and Comparative Example 3 (neither using Example 1 nor Example 2) on alleviation of wrinkles were determined before and after application to 30 subjects (women aged 20 to 35) for 10 weeks through instrumental measurement (cutometer SEM 575, C+K Electronic, Germany).

Table 3 below exhibits the effects of alleviating skin wrinkles. Preparation Example 1 was applied to the faces of Group A, Preparation Example 2 was applied to the faces of Group B, and Comparative Example 3 was applied to the faces of Group C.

TABLE 3

| | Effect of alleviating wrinkles (%) | | | | | Average (%) |
|---|---|---|---|---|---|---|
| Group A | 41.3 | 42.3 | 38.1 | 28.4 | 40.4 | 38.12 |
| Group B | 40.2 | 39.5 | 41.7 | 40.9 | 42.6 | 40.98 |
| Group C | 31.5 | 21.4 | 35.1 | 25.4 | 40.5 | 30.78 |

As can be seen from Table 3, the cosmetic compositions Preparation Example 1 using the low-molecular-weight peptide from Citrus junos seeds (Example 1) and Preparation Example 2 using the low-molecular-weight anionic peptide from Citrus junos seeds (Example 2) exhibited excellent effects of alleviating wrinkles compared to Comparative Example 3, and in particular, it can be seen that Preparation Example 2 using the low-molecular-weight anionic peptide from Citrus junos seeds (Example 2) exhibited better effects. In other words, it can be seen that the cosmetic composition having an excellent effect of alleviating skin wrinkles may be prepared when the low-molecular-weight anionic peptide from Citrus junos seeds of the present invention is used.

As such, it can be seen that the low-molecular-weight anionic peptides from Citrus junos seeds of the present invention and the cosmetic composition containing the same are effective in reducing fine dust adsorption, skin irritation, and skin wrinkles. That is, the low-molecular-weight anionic peptide derived from Citrus junos seeds of the present invention exhibits a negatively charged surface potential and thus can effectively reduce the adsorption of fine dust based on electrostatic repulsion with fine dust. According to the present invention, using the low-molecular-weight anionic peptide derived from Citrus junos seeds, it is possible to prepare a cosmetic composition having excellent effects of alleviating skin irritation and skin wrinkles caused by an increase in inflammatory cytokines due to fine dust.

1. A method for preparing a composition containing a low molecular weight anionic peptide, the method comprising:
(a) immersing a Citrus junos seed powder in purified water;
(b) inducing a primary enzymatic reaction using a subtilisin enzyme derived from Bacillus subtilis after the immersion in step (a);
(c) inducing a secondary enzymatic reaction using pepsin after the primary enzymatic reaction in step (b);
(d) centrifuging the enzymatic reaction solution to recover a supernatant after the secondary enzymatic reaction in step (c);
(e) adding ethanol to the supernatant recovered in step (d) to induce a precipitation reaction and centrifuging the precipitate to obtain a supernatant; and
(f) separating a low-molecular-weight anionic peptide from the supernatant obtained in step (e) or a solution obtained by dissolving a powder obtained by lyophilizing the supernatant in distilled water using a cation exchange resin.

2. The method according to claim 1, wherein the low-molecular-weight anionic peptide derived from the Citrus junos seed has a molecular weight of 1,000 Da or less.

3. The method according to claim 1, wherein the primary enzymatic reaction of step (b) is performed at a pH of 6 to 8 and the secondary enzymatic reaction of step (c) is performed at a pH of 2 to 4.

4. The method according to claim 1, wherein the primary enzymatic reaction of step (b) is performed at a pressure of 20 to 100 bar and a temperature of 50 to 60° C., and
the secondary enzymatic reaction of step (c) is performed at a pressure of 20 to 100 bar and a temperature of 35 to 45° C.

5. A method of preventing fine dust adsorption on a skin of a subject in need thereof, the method comprising administering the composition prepared by the method according to claim 1 to the skin of the subject.

6. A method of alleviating skin irritation, the method comprising administering to a skin of a subject in need thereof the composition prepared by the method according to claim 1.

7. A method of alleviating skin wrinkles, the method comprising administering to a skin of a subject in need thereof the composition prepared by the method according to claim 1.
